(19) United States
(12) Patent Application Publication
(10) Pub. No.: US 2019/0231685 A1
(43) Pub. Date: Aug. 1, 2019

(54) ANTIHISTAMINE ORAL FILM DOSAGE FORM AND METHOD OF ADMINISTRATING SAME

(71) Applicant: Intelgenx Corp., St-Laurent (CA)

(72) Inventors: Nadine Paiement, St-Laurent (CA); Rodolphe Obeid, Pierrefonds (CA); Billal Tir, Montreal (CA)

(73) Assignee: Intelgenx Corp., St-Laurent (CA)

(21) Appl. No.: 16/261,284

(22) Filed: Jan. 29, 2019

Related U.S. Application Data
(60) Provisional application No. 62/623,691, filed on Jan. 30, 2018.

(51) Int. Cl. A61K 9/00; A61K 31/4402; A61K 31/138; A61K 47/12; A61K 47/38; A61K 47/32

(52) U.S. Cl. CPC A61K 9/0056; A61K 31/4402; A61K 47/32; A61K 47/12; A61K 47/38; A61K 31/138

(57) ABSTRACT

An oral film dosage form for administration in the buccal cavity includes a film layer containing a safe and effective amount of an antihistamine and 3% to 12% (w/w) of a numbness mitigating agent.

### CROSS-REFERENCE TO RELATED APPLICATIONS

This application claims priority to U.S. Provisional Application No. 62/623,691, filed Jan. 30, 2018, which is hereby incorporated by reference in its entirety.

### FIELD OF THE DISCLOSURE

This disclosure relates to oral film dosage forms for administering antihistamines and to treatment of medical conditions using antihistamines.

### BACKGROUND OF THE DISCLOSURE

Antihistamines are drugs which treat allergic rhinitis and other allergies Antihistamines can give relief when a person has nasal congestion, sneezing, or hives because of pollen, dust mites, or animal allergy. Typically people take antihistamines as an inexpensive, generic, over-the-counter drug with few side effects. Antihistamines are usually for short-term treatment. Chronic allergies increase the risk of health problems which antihistamines might not treat, including asthma, sinusitis, and lower respiratory tract infection. Doctors recommend that people talk to them before any longer term use of antihistamines.

Although typical people use the word “antihistamine” to describe drugs for treating allergies, doctors and scientists use the term to describe a drug that opposes the activity of histamine receptors in the body. Antihistamines are classified according to the histamine receptor that they act upon. The two largest classes of antihistamines are H₁-antihistamines and H₂-antihistamines. Antihistamines that target the histamine H₁-receptor are used to treat allergic reactions in the nose (e.g., itching, runny nose, and sneezing) as well as for insomnia. They are sometimes also used to treat motion sickness or vertigo caused by problems with the inner ear. Antihistamines that target the histamine H₂-receptor are used to treat gastric acid conditions (e.g., peptic ulcers and acid reflux). H₁-antihistamines work by binding to histamine H₁ receptors in mast cells, smooth muscle, and endothelium in the body as well as in the tuberomammillary nucleus in the brain; H₂-antihistamines bind to histamine H₂ receptors in the upper gastrointestinal tract, primarily in the stomach.

Histamine receptors exhibit constitutive activity, so antihistamines can function as either a neutral receptor antagonist or an inverse agonist at histamine receptor. Only a few currently marketed H₁-antihistamines are known to function as inverse agonists.

A problem encountered when formulating an oral film containing antihistamines is the undesirable sensation of numbness in the mouth generated by the presence of the active directly in contact with the mouth,

Quick disintegrating tablets are an appealing alternative dosage form to consumers. Orally disintegrating tablets are formulated to disintegrate and disperse in the oral cavity prior to release of the active ingredient. The active ingredient can then be swallowed and later absorbed through the GI system like traditional oral dosage forms. Alternatively, the active ingredient can dissolve in saliva and be absorbed via the oral mucosa directly into systemic circulation. Such formulations are able to circumvent the first-pass metabolism effect, which increases the bioavailability of active ingredients.

Brompheniramine is part of a series of antihistamines including pheniramine (Naphcon) and its halogenated derivatives and others including fluorpheniramine, chlorpheniramine, dexchlorpheniramine (Polaramine), triprolidine (Actifed), and iodopheniramine.

The halogenated alkylamine antihistamines all exhibit optical isomerism and brompheniramine products contain racemic brompheniramine maleate whereas dexbrompheniramine (Drixoral) is the dextrorotary (right-handed) stereoisomer.

Brompheniramine has antidepressant properties, inhibiting reuptake of the neurotransmitter serotonin and norepinephrine. Based on this knowledge, Arvid Carlsson and his colleagues, working at the Swedish company Astra AB, were able to derive the first marketed selective serotonin reuptake inhibitor, zimelidine, from brompheniramine.^{[2]}

Like other agents of this type, brompheniramine also has analgesic-sparing (potentiating) effects on opioid analgesics, commonly reducing codeine, dihydrocodeine, and hydrocodone requirements by 10 to 35 percent.

However, current antihistamines products such as diphenhydramine and brompheniramine, including disintegrating dosage forms, are formulated to either prevent dissolution in the oral cavity because it would cause an unpleasant bitter taste and numbing of the oral cavity or promote the dissolution of the tablet within the oral cavity to obtain some level of buccal absorption. It was disclosed that the biopharmaceutical properties of diphenhydramine at the pH conditions found in the oral cavity (pH 6.0 to 7.0) disfavors oral absorption when administered through quickly disintegrating tablet. As a result, diphenhydramine in these products is swallowed and absorbed through the GI system like traditional dosage forms, which leads to a delay in symptom relief or is alternatively quickly disintegrated in the oral cavity with a buffer system, that attempt to control the local pH to promote buccal absorption of the solubilized active. As such, there remains a need for improved options for fast acting antihistamine medications (e.g. diphenhydramine medications). In particular, there exists a need for a sublingual or buccal diphenhydramine composition that at least mitigate inconveniences encountered when administered via the oral cavity. Of particular concern are the bitterness taste of several antihistamines and numbness sensation felt once the active encounter the mucosa.

There is thus a need for an oral film dosage form that eases administration of the active pharmaceutical products (API) while mitigating numbness sensation associated with the contact between API and mucus membranes.

### SUMMARY OF THE DISCLOSURE

Disclosed is an oral film dosage form designed for improving oral administration of antihistamines such as brompheniramine and diphenhydramine.

The film layer can be configured for buccal transmucosal and oral delivery of the active agent(s).

In accordance with some aspects of this disclosure, a film dosage form is configured for oral administration of numbness inducing antihistamines.

According to certain aspects of this disclosure, an antihistamine oral film dosage form is configured to reduce numbness sensation generally associated with contact between antihistamines and oral mucus membranes.

Also disclosed is a method of administering antihistamines to mitigate the undesirable numbness sensation associated with the oral administration of antihistamines (e.g., buccally or sublingually). The film layer is configured for buccal, transmucosal and/or sublingual delivery of the active agent. These and other features, advantages and objects of the various embodiments will be better understood with reference to the following specification and claims.

### BRIEF DESCRIPTION OF THE DRAWINGS

FIG. 1 is a graph showing average dissolution time for film with brompheniramine actives (percentage along the vertical axis and time in minutes along the horizontal axis).

### DETAILED DESCRIPTION OF ILLUSTRATIVE EMBODIMENTS

Quick disintegrating antihistamines products are known. However, current disintegrating antihistamines products such as diphenhydramine products do not sufficiently dissolve in saliva to be absorbed in the oral cavity. Instead, consumers generally swallow the disintegrated tablet along with saliva and antihistamines such as diphenhydramine passes into the GI system, where traditional absorption processes occur. Other known products are quick disintegrating antihistamines tablet such as those containing diphenhydramine which allegedly result in adequate quick disintegration. Quick disintegration of antihistamine tablet often results in a bitter aftertaste with the presence of an unwelcomed numbing effect while also having limited buccal absorption. Quickly disintegrating tablet still result in a substantial portion of the API being swallow and ingested through the GI track where traditional absorption processes occur. Known disintegrating antihistamine product are offered in traditional swallow or quick disintegration tablet dosage form. No antihistamine oral film product is known. It is hypothesized that the lack of antihistamines oral films is at least in part a consequence of the bitter aftertaste and numbing sensation sustained following oral administration of antihistamine, principally brompheniramine (bromferamine . . . ) and diphenhydramine via oral film dosage forms.

Though prior art document such as U.S. patent pub. No. 2017/0172948 identified some antihistamines such as diphenhydramine as being readily absorbed across buccal tissue only at a narrow pH ranges (pH of 7.0 to 8.0) which require complicated buffering systems, absorption dynamics and bioavailability of actives administered via quick disintegrating or quick dissolving tablet differs significantly from those administered through buccal and/or sublingual films. The former requiring adequate pH for disintegration and/or dissolution of the tablet in the saliva which result in all the active being available in soluble form at the same time while the latter (the oral film) has the active progressively solubilizing and optimally being absorbed progressively while becoming solubilized in the saliva. Oral film dosage form administration by design are placed within the oral cavity directly in contact with the oral mucosa or sublingual mucosa, thus promoting buccal absorption. In addition, prolonged residence time of buccal and/or sublingual absorption containing antihistamines promote buccal or sublingual absorption thus favoring bypass of first-pass metabolism and resulting in higher bioavailability of the active and therefore requires less stringent conditions for buccal absorption when compared with quickly dissolving/disintegrating tablets of the same loading.

It is believed that increased absorption from administration of antihistamines through buccal or sublingual film result in faster onset of action while the present oral film dosage form allows mitigation of the bitterness/numbness associated with orally administered antihistamines when in contact with the mucosa under a solubilized form. It was found that the numbness sensation experienced as a result of oral administration of antihistamines may be substantially reduced by administering an oral film dosage form comprising at least about 3% (w/w) to about 12% (w/w) of citric acid. The high concentration of citric acid with the presence of a taste marker was found to significantly diminish the bitter aftertaste while mitigating to a large extent the undesired numbness sensation. The selected potent flavors have a taste that competes with bitter after taste of the API while bitter masker and sweetener taste compete with the bitterness of the API in the mouth.

Also presently disclosed is a method for inducing sleep or providing allergy relief in subjects in need thereof. The method generally comprises placing an oral film dosage form in the oral cavity of a subject, the oral dosage form including antihistamines such as Brompheniramine or diphenhydramine or a pharmaceutically acceptable salt thereof.

According to one embodiment, the method comprises placing an oral film dosage form in the oral cavity of a subject, the oral dosage form including antihistamines such as Brompheniramine or diphenhydramine or a pharmaceutically acceptable salt thereof in combination with at least about 3 percent (w/w) citric acid. According to the disclosed method, the antihistamine administered via a dosage form that maximize the contact between the mucosa and the surface of the dosage form.

Oral films by their nature are thin and present a significant surface/volume ratio thus mitigating losses associated with GI track absorption and increases in bioavailability of the antihistamines administered using the method disclosed herein.

According to embodiments, the disclosed method for inducing sleep or providing allergy relief in subjects in need thereof comprises placing an oral film dosage form comprising at least 3%(w/w) of numbness mitigation additive (i.e. citric acid) in the oral cavity of a subject. In one example, the method includes administering the oral dosage form sublingually. According to some embodiment, the antihistamine oral film comprises from about 3% to about 12% (w/w) numbness mitigation additive.

In one example, the present invention relates to a method for enhancing oral absorption of diphenhydramine. The method can comprise placing an oral film dosage form in the oral cavity of a subject in need of such treatment. The oral film dosage form comprises a safe and therapeutically effective amount of diphenhydramine or a pharmaceutically acceptable salt thereof in combination and a polymeric matrix that allows the diphenhydramine oral film that remains in contact with the mucosa For at least a period of two minutes. The oral film dosage form containing the diphenhydramine thus takes at least a period of 3.5 minutes to entirely disintegrate or dissolve thereby increasing the ability of the dosage form to deliver the drug transmucosally.

As used herein, “absorption” means penetration of the active ingredient through the oral mucosa (i.e. buccal or sublingual mucosa) and into systemic circulation.

As used herein, “bioavailability” refers to a rate and extent to which the active ingredient or therapeutic moiety is absorbed from a drug product and becomes available for therapeutic action. In one example the active ingredient can be diphenhydramine and it can reach the systemic circulation and can be available at its site of action.

As used herein, “disintegrate” or “disintegrating” means the process whereby an oral dosage form falls apart into smaller aggregates or particles.

As used herein, “dissolve” or “dissolving” means the process whereby a solid becomes incorporated into a liquid so as to form a solution.

As used herein, the term “permeability enhancer” is a material capable of decreasing the penetration barrier of the oral mucosa and enhancing permeation of antihistamines through the oral mucosa.

As used herein, “subject” means animals or humans.

As used herein, “therapeutically effective amount” of an active ingredient refers to a non-toxic but sufficient amount of the active ingredient to provide the desired sleep effect and/or response to allergic reaction. The amount of active ingredient that is “effective” will vary from subject to subject, depending on the age and general condition of the individual, the particular active agent or agents, and the like.

As used herein, the articles “a” and “an” are understood to mean one or more of the material that is claimed or described, for example, “a permeability enhancer”.

As used herein, the term “antihistamines” encompasses Brompheniramine, Cetirizine, Chlorpheniramine, Clemastine, Diphenhydramine, Fexofenadine, Loratadine, Terfenadine, triprolidine, carbinoxamine, promethazine, loratadine, desloratadine dexchlorpheniramine, levocetirizine, hydroxyzine, and triprolidine. Other pharmaceutically active agents having a comparable antihistamine effect are also encompassed.

All weights, measurements and concentrations herein are measured at 23 degrees Celsius (° C.) and 50% relative humidity, unless otherwise specified.

All percentages, parts and ratios as used herein are by weight of the total oral dosage form, unless otherwise specified. All such weights as they pertain to listed ingredients are based on the active level and, therefore do not include solvents or by-products that may be included in commercially available materials, unless otherwise specified.

The antihistamines oral film dosage form comprises from about 10 mg to about 45 mg of antihistamines. In one example, the antihistamines oral film dosage form comprises from about 10 mg to about 45 mg of diphenhydramine, diphenhydramine HCl or any acceptable salt thereof.

Oral films suitable for use in preparing the disclosed dosage forms are typically comprised of at least one water soluble polymer. In certain embodiments, the disintegrating film does not include insoluble polymers or other materials that can leave a gritty, unpleasant residue. Surfactants, polyalcohols, and or plasticizers may be incorporated into the disintegrating film to facilitate or enhance wettability and disintegration of the film. The film-forming polymer or combination of film-forming polymers can comprise 20% to 80% or 30% to 70% of the weight of the film oral dosage form on a dry basis.

Water soluble polymers that can be employed in the disclosed films include water soluble cellulose derivatives, including hydroxypropylmethyl cellulose, hydroxyethyl cellulose, hydroxypropyl cellulose; polyvinyl pyrrolidone (PVP); copovidone (a copolymer of 1-vinyl-2-pyrrolidone and vinyl acetate); other copolymers of vinyl pyrrolidone; other polymers or copolymers of substituted vinyl pyrrolidone; derivatives of polyvinyl pyrrolidone; polyethylene oxide, carboxymethyl cellulose; polyvinyl alcohol; natural gums, including xanthan, tragacanth, guar, acacia and arabic gums; and water soluble polyacrylates. Combinations of these water soluble polymers or other water soluble polymers can also be used. Examples of substituted vinyl pyrrolidones include N-vinyl-3-methyl-2-pyrrolidone, N-vinyl-4-methyl-2-pyrrolidone, N-vinyl-5-methyl-2-pyrrolidone, N-vinyl-5,5-dimethyl-2-pyrrolidone, N-vinyl-3,3,5-trimethyl-2-pyrrolidone and others. Examples of monomers that can be copolymerized with vinyl pyrrolidone or substituted vinyl pyrrolidones include vinyl aromatic monomers such as styrene, and acrylate or methacrylate monomers such as methyl methacrylate and 2-dimethylaminoethyl methacryl ate.

The terms “surfactant” and “polyalcohol” are intended to have their ordinary meanings. Specifically, the term “surfactant” is intended to mean an amphophilic compound that lowers the surface tension of a liquid, the interfacial tension between two liquids, or the interfacial tension between a liquid and a solid. Examples of surfactants that can be used in a disintegrating film of an oral dosage form are known and include polyoxy-ethylene sorbitan fatty acid esters, an α-hydro-co-hydroxypoly (oxyethylene) poly (oxypropylene) poly(oxyethylene) block copolymer, a polyoxyethylene allyl ether, a polyoxyethylene or a castor oil derivative. Combinations of surfactants can be used. The term “polyalcohol” means a sugar alcohol, which is a hydrogenated form of a carbohydrate having a carbonyl group that has been reduced to a primary or secondary hydroxyl group. Polyalcohols are also distinguishable based on their chemical formula. Polyalcohols have the general formula H(HCHO)n+1H, whereas sugars have the general formula H(HCHO)n HCO. Common examples of polyalcohols or sugar alcohols that can be used from the disclosed films include glycol, glycerol, erythritol, threitol, arabitol, xylitol, ribitol, mannitol, sorbitol, galactitol, fucitol, iditol, inositol, volemitol, isomalt, maltitol, lactitol, maltotritol and maltotetraitol.

According to one embodiment, the antihistamine oral film dosage form may be cast from a water based formulation or from an organic based formulation. In one example, the antihistamine oral film comprises diphenhydramine suspended in a non-alcohol organic solvent based formulation. In one example, the diphenhydramine is suspended in a methyl ethyl ketone solvent based formulation

Because of the taste of antihistamines such as brompheniramine and diphenhydramine, which is generally perceived as unpleasant in addition to produce a numbness sensation when in contact with the mucosa, it is beneficial to add a sweetener, flavoring agent, refreshing agent, taste-masking agent, or a combination of these materials. Examples of sweeteners that can be used in the disclosed antihistamines film dosage forms include acesulfame potassium, aspartame, aspartan-acesulfame salt, cyclamate, erythritol, glycerol, glycyrrhizin, hydrogenated starch hydrolysate, isomalt, lactitol, maltitol, mannitol, neotame, polydextrose, saccharin, sorbitol, sucralose, tagatose, xylitol, dextrose, glucose, fructose, and honey. Flavoring agents that can be added to the disclosed antihistamines film dosage forms include isoamyl acetate (banana flavor), benzaldehyde (cherry flavor), cinnamaldehyde (cinnamon flavor), ethyl propionate (fruit flavor), methyl anthranilate (grape flavor), limonene (orange flavor), ethyl decadienoate (pear flavor), allyl hexanoate (pineapple flavor), ethyl meltol, ethylanillin (vanilla flavor), and methyl salicylate (wintergreen flavor). Refreshing agents, also called cooling agents, are chemicals that trigger the cold sensitive receptors creating a cold sensation. Refreshing agents that can be added to the antihistamines film oral dosage forms disclosed herein include menthol, thymol, camphor and eucalyptol.

Sweeteners, flavoring agents, and refreshing agents can be added in quantities, generally up to a total amount of 5% to 10% of the weight of the film on a dry basis, e.g., 0.1% to 10%, or 0.5% to 5%.

According to some embodiments, the antihistamines film oral dosage form comprises numbness mitigation additive such as citric acid in quantities up to about 12% of the weight of the film on a dry basis, e.g., about 3% to about 12%, or about 6% to about 10%.

According to some embodiments, the antihistamines film oral dosage form has an active to numbness mitigating agent (citric acid) ratio varying from about 3 to about 12, preferably about 5 to about 10.

The antihistamines film oral dosage forms disclosed herein can advantageously employ an antioxidant or oxygen scavenger to prevent or reduce oxidative degradation of the antihistamines salt, free base or prodrug prior to use. Examples of oxygen scavengers or antioxidants that substantially improve long-term stability of an antihistamines film oral dosage form against oxidative degradation of the active agent are sulfite salts such as sodium sulfite, sodium bisulfite, sodium metabisulfite and analogous salts of potassium and calcium. A suitable amount of salt (e.g., sodium sulfite) is from about 0.01% to 5% or 0.1% to 1% of the weight of the film on a dry basis.

In order to promote adhesion of the antihistamines film oral dosage form to oral mucosa, it is advantageous to add a mucoadhesive agent to the film product. Examples of mucoadhesive agents that can be added to the antihistamines film oral dosage form to promote adhesion to oral mucosa include sodium alginate, sodium carboxymethyl cellulose, guar gum, hydroxyethyl cellulose, hydroxypropyl cellulose, hydroxypropylmethyl cellulose, karya gum, methylcellulose, polyethylene oxide, retene and tragacanth. Such mucoadhesive agent may be added to the film formulation in an amount of from about 0.5% to about 20%, or about 1% to about 5%), of the total weight of the film on a dry basis.

In certain embodiments, the disclosed films may include a plasticizer. Plasticizers can be advantageously employed in the film formulations as needed to suitably modify the flexibility of the film to facilitate processing and allow the film to easily conform to the shape of the oral mucosa to which the film is applied. Plasticizers that can be effectively employed in the disclosed antihistamines film oral dosage forms to improve flexibility of the film include ethylene glycol, propylene glycol, tributyl citrate, tri ethyl citrate and glycerol. Depending on the selected film-forming polymers and other components of the film formulation, a suitable amount of plasticizer is typically from about 0.1% to 10%, 0.5% to 5%, or 1% to 5%.

According to one embodiment, some antihistamines such as diphenhydramine behave as a plasticizer when dissolved in water based oral film formulations. Accordingly, it is disclosed a water based antihistamine film oral dosage forms that is free or substantially free of plasticizer. The term “substantially free” of plasticizer means that the film oral dosage forms contain no deliberately added plasticizer, and any unavoidable or incidental plasticizer are only present, if at all, as impurities in other ingredients in amounts that do not affect measurable properties relating to film plasticity, dissolution or disintegration rates by more than 10%, and do not adversely affect measurable stability properties. The term “free” of plasticizer means that incidental or unavoidable plasticizer impurities are present in only inconsequential amounts that affect film plasticity, film dissolution rate by less than 1%. In certain embodiments, the presence of plasticizer are each limited to less than 1000 ppm (w/w), less than 500 ppm (w/w), less than 100 ppm (w/w), less than 40 ppm (w/w), or less than 10 ppm (w/w).

According to one embodiment, titanium dioxide can be added in amounts from about 0.05% to 5%, 0.1% to 3%, or 0.5 to 2% of the weight of the film. The titanium dioxide acts as a disintegrant in the disclosed films, as well as a texture modifier that improves mouth feel, and an opacify or coloring agent. This amount is effective for increasing the rate at which the film will dissolve in an aqueous medium. The increased rate of disintegration can be at least 5%, at least 10%, at least 20%, at least 50% or at least 75%.

Multilayer, multilayer with active, multilayer with placebo, unidirectional multilayer mitigate contact between the tongue and the active, multilayer for sublingual with varying thickness, wherein the thickness of the multilayer is proportional to the absorption of eitherside of the sublingual mucosa. Wherein the multilayer comprises a thin placebo layer in between the upper and lower active layer to prevent or at least mitigate active from the upper layer to be absorbed by the buccal mucosa or opposite, having active from the bottom layer migrating to the upper layer to be absorbed from the mucosa below the tongue. Optimization of the layer thickness mitigate the unwanted numbness by promoting absorption of either the upper and lower layer by their respective mucosa and hence limiting any unabsorbed active to circulated within the mouth.

According to embodiment, the oral film dosage form may comprise several layer based on the required loading. Subsequent passage coating may increase the potential loading contained within a film. Subsequent passage coating process also facilitate the manufacture of highly viscous blend such as aqueous or water based diphenhydramine film formulations. As such, the multilayer oral film dosage form may comprise a plurality of layer in an amount that varies depending on the desired film loading.

In one example, a first film layer comprises from 5 to 15 mg of antihistamine (e.g. diphenhydramine), onto which a second film layer which also comprises from 5 to 15 mg of antihistamine (which may be the same as in the first layer or may be a different antihistamine than the one comprised in the first layer). A multilayer antihistamine is especially suitable for multi active films. The process of making the film by coating a film layer on top of a previously coated layer allows for multilayer oral film with a plurality of actives which may require different film physical properties. Multilayer antihistamine oral films are also especially suitable when different actives are required which have different absorption profile, and which may require different chemical condition for optimal absorption.

Multilayer with directional absorption and second layer with citric acid to mitigate numbness. According to this embodiment, the antihistamine oral film dosage form comprises a first layer comprising the antihistamine active and a second layer Loading dependent multilayer

Disclosed is a method of administering a multilayer directional film having the numbness mitigating element, the method comprising the step of:
a. Administering (bucally or sublingually the film)
b. indicate the orientation (which side of the film which has to be applied to the mucosa and
   where the active is in contact with the mucosa while the numbness mitigating agent and flavor is provided on a side opposite to the side of the film that is in contact with the mucosa.
   This way, one side of the film (active side or active plus numbness mitigating agent) is in contact with the mucosa while the opposite side (flavor and numbness mitigating agent) is in contact with the saliva and thus mitigate the undesirable numbness effect relater to administration of antihistamines.

According to some embodiments, the disclosed oral film dosage from is cast from a nonalcoholic organic solvent based formulation.

According to some embodiment, a method of forming a film of the present disclosure includes combining the various ingredients in generally any order, employing water, a combination of water and water-miscible solvents such as lower alcohols (e.g., ethanol) or organic solvents alone or as a mixture. For example, the plasticizer and additives (e.g., sweetening agents, colorants, flavorants, and opacifying agents) can be dissolved or dispersed in a sufficient amount of solvent that is agitated to form a homogenous solution or suspension to which the water soluble polymer(s) is (are) added. Heat, vacuum and agitation may be applied as needed during addition of the water soluble polymer until a homogenous solution or homogenous suspension is obtained. Thereafter, the active ingredient(s) is (are) added, and the solution or suspension is cast or coated onto a carrier material and dried to form a film. Examples of suitable carrier materials include non-siliconized polyethylene terephthalate film, non-siliconized kraft paper, polyethylene-impregnated kraft paper and non-siliconized polyethylene film. The liquid film composition can be coated onto the carrier material using generally any conventional coating equipment, including knife-over-roll, extrusion die, reverse roll, or Meyer roll coating equipment.

Upon drying, the resulting solid film can have a thickness of generally 5 to 350 μm, such as 10 to 300 μm, 20 to 200 μm or 20 to 100 μm. The film can be cut into individual pieces having a suitable size to facilitate administration of a targeted dosage of active agent(s).

The composition and methods of the present invention can comprise, consist of, or consist essentially of, the essential elements and limitations of the invention described herein, as well as any additional or optional ingredients, components, or limitations described herein or otherwise useful in personal health care articles intended for use or consumption by a subject.

Buccal and sublingual antihistamine dosage forms may advantageous comprise a penetration enhancer that promotes absorption of antihistamines salt, free-base or prodrug through oral mucosa and reduce the amount of antihistamines that is introduced into the gastrointestinal tract. In order to increase the passage of diphenhydramine through the oral mucosa, one or more permeability enhancers may be used. Any permeability enhancers effective in increasing oral permeability may be used. Non-limiting examples of permeability enhancers include bile salts, surfactants, synthetic surfactants, cyclodextrins, solvents, and combinations thereof. In one example, the permeability enhancer can be sodium dodecyl sulfate, polyethylene glycol (PEG)-8 stearate (commercially available from Croda, Inc., Edison, N.J., USA), citrate buffer, oleic acid, sodium caprate, cetylpyridinium chloride, menthol, and combinations thereof. In one example, the permeability enhancer can be sodium caprate, cetylpyridinium chloride, and combinations thereof. In one example, the permeability enhancer can be sodium caprate. In one example the permeability enhancer can be cetylpyridinium chloride. In one example, the permeability enhancer cannot be citrate, oleic acid, sodium dodecyl sulfate, PEG-8 stearate or any permeation enhancer that buffers the pH of the oral film when administered. A penetration enhancer, such as the ones mentioned above, can be added to the antihistamines film oral dosage form in an amount of from about 0.1% to about 10%, 0.5% to 5%, or 1% to 3% of the weight of the film dosage form on a dry basis to significantly enhance absorption of antihistamines from the film through oral mucosa.

In one example the oral dosage form can contain about 0.1% to about 10% permeability enhancer, in another example from about 0.25% to about 8%, in another example from about 0.3% to about 6%, in another example from about 0.5% to about 3%, and in another example from about 0.75% to about 1.5%. In one example, the oral dosage form can contain about 1% permeability enhancer.

In one example, the ratio surface to volume of the antihistamine is from 40 to 500. In one example, the ratio of surface area to the volume (surface/volume ratio) of the diphenhydramine oral film dosage form is 40 to 500, preferably from 50 to 200. In one example, the surface to volume ratio of a multilayer diphenhydramine buccal film is from 40 to 500. In one example, the surface area to volume ratio of a sublingual film having two surfaces of contact (e.g. where the active may be absorbed, either a single layer film or multilayer film where both outer layers comprise diphenhydramine active) is from 80 to 1000. In one example, the oral film dosage form has surface to volume ratio greater than 50.

In one example, the oral dosage form can disintegrate in about 3.5 minute to about 15 minutes, in another example in about 5 minutes to about 12 minutes. Disintegration can be measured according to the Disintegration Method described hereafter.

In one example, the diphenhydramine in the oral dosage form can be completely dissolved within about 3.5 minutes, in another example within about 5 minutes, in another example within about 7 minutes, in another example within about 10 minutes, in another example within about 12 minutes, and in another example within about 15 minutes. Dissolution can be considered complete when 90% of diphenhydramine in the dosage form is dissolved. Dissolution can be measured according to the Dissolution Method described hereafter. According to one example, the antihistamine may be designed to last for a longer period to allow continuous administration of the active over a prolonged period of time, thus extending the period of treatment.

### EXAMPLES

Organic solvent based (suspended active within the oral film) diphenhydramine (DPH) film oral dosage forms are prepared using the above described processes and compositions listed in the following Tables.

TABLE 1

| Ex 1 (DPH) | g | % wet | % dry |
|---|---|---|---|
| DPH | 12.00 | 7.87 | 31.96 |
| HPC L | 22.00 | 14.42 | 58.59 |
| MEK | 115.00 | 75.39 | NA |
| hpmc | 1.25 | 0.82 | 3.33 |
| citric acid | 1.00 | 0.66 | 2.66 |
| sucralose | 0.30 | 0.20 | 0.80 |
| Cherry | 0.30 | 0.20 | 0.80 |
| flavor | | | |
| D&C red | 0.30 | 0.20 | 0.80 |
| #28 | | | |
| TiO2 | 0.40 | 0.26 | 1.07 |
| Total | 152.55 | 100 | 100 |
| Total Dry | 37.55 | | |

DPH/Citric Acid ratio 12.0

TABLE 2
Ex 2 (DPH)

| | | | |
|---|---|---|---|
| DPH | 14.00 | 9.43 | 41.8 |
| Klucel HPC L | 15.00 | 10.10 | 44.8 |
| MEK | 115.00 | 77.44 | NA |
| HPMC | 1.20 | 0.81 | 3.58 |
| sucralose | 0.30 | 0.20 | 0.9 |
| Cherry | 0.30 | 0.20 | 0.9 |
| flavor | | | |
| violet | 0.30 | 0.20 | 0.9 |
| TiO2 | 0.40 | 0.27 | 1.19 |
| Citric acid | 2.00 | 1.35 | 5.97 |
| Total | 148.50 | 100 | 100 |
| Total dry | 33.50 | | |

DPH/Citric Acid ratio 7.0

TABLE 3
Ex 3 (DPH)

| | | | |
|---|---|---|---|
| DPH | 14.00 | 9.36 | 40.6 |
| Klucel HPC L | 15.00 | 10.03 | 43.5 |
| MEK | 115.00 | 76.92 | |
| HPMC | 1.20 | 0.80 | 3.48 |
| sucralose | 0.30 | 0.20 | 0.87 |
| Cherry | 0.30 | 0.20 | 0.87 |
| flavor | | | |
| violet | 0.30 | 0.20 | 0.87 |
| TiO2 | 0.40 | 0.27 | 1.16 |
| Citric acid | 3.00 | 2.01 | 8.7 |
| Total | 149.50 | 100 | 100 |
| Total dry | 34.50 | | |

DPH/Citric Acid ratio 4.7

TABLE 4
Ex 4 (DPH)

| | | | |
|---|---|---|---|
| DPH | 14.00 | 9.30 | 39.4 |
| Klucel HPC L | 15.00 | 9.97 | 42.3 |
| MEK | 115.00 | 76.41 | |
| HPMC | 1.20 | 0.80 | 3.38 |
| sucralose | 0.30 | 0.20 | 0.85 |
| Cherry | 0.30 | 0.20 | 0.85 |
| flavor | | | |
| violet | 0.30 | 0.20 | 0.85 |
| TiO2 | 0.40 | 0.27 | 1.13 |
| Citric acid | 4.00 | 2.66 | 11.3 |
| Total | 150.50 | 100 | 100 |
| Total dry | 35.50 | | |

DPH/Citric Acid ratio 3.5

Water based (dissolved active within the oral film) diphenhydramine film oral dosage forms are prepared using the above described processes and compositions listed in the following Tables.

TABLE 5

| | | |
|---|---|---|
| | | % |
| (g) | % wet | dry |

| | | | |
|---|---|---|---|
| DPH | 12.00 | 10.90 | 31.08 |
| PEO 200 | 12.00 | 10.90 | 31.08 |
| Water | 75.00 | 68.13 | |
| HPMC E50 | 1.15 | 1.04 | 2.978 |
| HPMC E5 | 6.90 | 6.27 | 17.87 |
| Citric acid | 2.00 | 1.82 | 5.179 |
| sucralose | 0.30 | 0.27 | 0.777 |
| Cherry flavor | 0.30 | 0.27 | 0.777 |
| FD&C red #3 | 0.03 | 0.03 | 0.078 |
| TiO2 | 0.40 | 0.36 | 1.036 |
| total | 110.08 | | |
| Total Dry | 35.08 | | |

DPH/Citric Acid ratio 6.0

TABLE 6

| | | | % |
|---|---|---|---|
| | (g) | % wet | dry |
| DPH | 12.00 | 10.24 | 33.49 |
| HPC L | 12.50 | 10.67 | 34.89 |
| Water (38.5%) | 31.33 | 26.74 | |
| Methanol | 50.00 | 42.68 | |
| (61.5%) | | | |
| PVP K30 | 6.00 | 5.12 | 16.75 |
| Plasdone s630 | 1.80 | 1.54 | 5.024 |
| hpmc | 1.00 | 0.85 | 2.791 |
| Citric acid | 1.50 | 1.28 | 4.186 |
| sucralose | 0.30 | 0.26 | 0.837 |
| Cherry flavor | 0.30 | 0.26 | 0.837 |
| FD&C red #28 | 0.03 | 0.03 | 0.084 |
| TiO2 | 0.40 | 0.34 | 1.116 |
| total | 117.16 | | |
| Total Dry | 35.83 | | |

DPH/Citric Acid ratio 8.0

TABLE 7

| | | | % |
|---|---|---|---|
| | (g) | % wet | dry |
| DPH | 12.00 | 10.75 | 32.80 |
| HPC Nisso | 12.50 | 11.20 | 34.16 |
| Water (15.73%) | 11.80 | 10.57 | |
| Methanol | 63.20 | 56.64 | |
| (84.27%) | | | |
| PVP K30 | 6.00 | 5.38 | 16.40 |
| Plasdone s630 | 1.80 | 1.61 | 4.92 |
| HPMC | 1.26 | 1.13 | 3.44 |
| Citric acid | 2.00 | 1.79 | 5.47 |
| sucralose | 0.30 | 0.27 | 0.82 |
| Cherry flavor | 0.30 | 0.27 | 0.82 |
| FD&C red #28 | 0.03 | 0.03 | 0.08 |
| TiO2 | 0.40 | 0.36 | 1.09 |
| total | 111.6 | | |
| Total Dry | 36.59 | | |

DPH/Citric Acid ratio 6.0

Water based (dissolved active within the oral film) brompheniramine film oral dosage forms are prepared using the above described processes and compositions listed in the following Tables.

TABLE 8

| | | % wet | % dry |
|---|---|---|---|
| BP | 0.22 | 0.54 | 1.41 |
| DM | 1.1 | 2.71 | 7.05 |
| PE | 3.3 | 8.13 | 21.14 |
| water | 25 | 61.56 | |
| citric acid | 0.5 | 1.23 | 3.20 |
| fibersol Ag | 1 | 2.46 | 6.41 |
| hpmc k100 | 0.75 | 1.85 | 4.80 |
| eudragit EPO fresh | 1 | 2.46 | 6.41 |
| PEO300k | 2 | 4.92 | 12.81 |
| peo200K | 4 | 9.85 | 25.62 |
| cherry Evospray | 0.5 | 1.23 | 3.20 |
| fxx10181BF | | | |
| BITTER MASKER 686559 | 1 | 2.46 | 6.41 |
| ADVANTAME | 0.24 | 0.59 | 1.54 |
| Total wet | 40.61 | 100 | |
| total dry | 15.61 | | 100.00 |

TABLE 9

| | | % wet | % dry |
|---|---|---|---|
| BP | 0.22 | 0.54 | 1.37 |
| DM | 1.10 | 2.68 | 6.83 |
| PE | 3.30 | 8.03 | 20.48 |
| water | 25.00 | 60.81 | |
| citric acid | 1.00 | 2.43 | 6.21 |
| fibersol Ag | 1.00 | 2.43 | 6.21 |
| hpmc k100 | 0.75 | 1.82 | 4.66 |
| eudragit EPO fresh | 1.00 | 2.43 | 6.21 |
| PEO300k | 2.00 | 4.86 | 12.41 |
| peo200K | 4.00 | 9.73 | 24.83 |
| cherry Evospray | 0.50 | 1.22 | 3.10 |
| fxx10181BF | | | |
| BITTER MASKER 686559 | 1.00 | 2.43 | 6.21 |
| ADVANTAME | 0.24 | 0.58 | 1.49 |
| Total wet | 41.11 | 100.00 | |
| total dry | 16.11 | | 100.00 |
| Targer w | 146.4545 | | |

Dissolution Testing:

In the examples the following USP dissolution apparatus II with 500 mL dissolution vessels and paddle stirrers was used to perform the dissolution testing:

Paddle over disc of 56 mm and mesh #40 system is used

Distek Evolution 6300 Dissolution bath

Heater/Circulator TCS-0200C

Testing was conducted using 500 mL of 0.5% SLS dissolution media at 37° C. which is effective in discriminating between different formulations.

The materials used for preparation of dissolution media were: sodium lauryl sulfate (SLS) and water.

RO water from in-house Millipore Milli-Q Advantage AIO water system.

Dissolution results were measured as the mean of 3 to 6 replicates.

In formulations containing brompheniramine, each of the three active concentrations were measured using an HPLC Water 2695 separation module and waters 2996 photodiode array (PDA) detector set at an appropriate wavelength. For each drug, the optimal wavelength was selected after running UV scans in the dissolution medium.

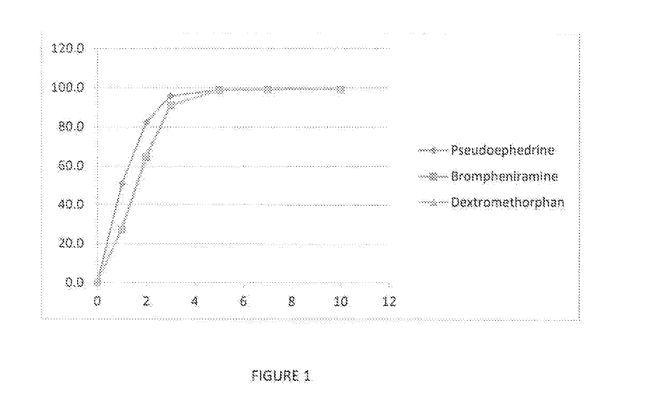

Referring to FIG. 1, the dissolution time of the film was about 5 minutes. The preferred dissolution time for antihistamine oral film dosage form is between about 3 to about 15 minutes.

The present disclosure further relates to a package for a pharmaceutical composition including a packaging material and an oral film dosage form for sublingual delivery of diphenhydramine contained within the packaging material. The oral dosage form can include diphenhydramine or a pharmaceutically acceptable salt thereof; a permeability enhancer without the presence of a buffering agent. The packaging material can include usage indicia that indicate the steps of (i) placing the oral dosage form under a subject's tongue, (ii) holding the oral film dosage form under the tongue for more than about 3.5 minutes, (iii) optionally avoiding eating, drinking, and/or talking in order to keep the oral film dosage form in place, (iv) optionally administering the oral dosage form at night; (v) optionally administering the oral dosage form in response to an allergic reaction, and/or (vi) optionally taking the oral dosage form every 4 to 6 hours as needed.

The above description is considered that of the preferred embodiment(s) only. Modifications of these embodiments will occur to those skilled in the art and to those who make or use the illustrated embodiments. Therefore, it is understood that the embodiment(s) described above are merely exemplary and not intended to limit the scope of this disclosure, which is defined by the following claims as interpreted according to the principles of patent law, including the doctrine of equivalents.

1. An oral film dosage form for administration in the buccal cavity, comprising: a film layer;
a safe and effective amount of an antihistamine incorporated into the film layer: and
from about 3 to 12% (w/w) of numbness mitigating agent.

2. The oral film dosage form of claim 1 wherein the numbness mitigating agent is citric acid.

3. The oral film dosage form of claim 1 wherein the dosage surface to volume ratio is between 40 to 1000.

4. The oral film dosage form of claim 1 wherein the dosage surface to volume ratio is between 50 to 500.

5. A pharmaceutical composition in unit dosage form formulated for buccal administration, wherein said unit dosage form is a film comprising:
(a) a layer comprising:
(b) from 20 to 40% (w/w) diphenhydramine;
(c) from 60 to 45% (w/w) a film former polymer having selected from hydroxypropyl cellulose, hydroxypropyl methyl cellulose, hydroxyethyl cellulose, carboxymethyl cellulose, methyl cellulose, polyvinylpyrollidone, pectin, gelatin, sodium alignate, polyvinyl alcohol, maltodextrins, Eudragit, and pullulan; and
(d) from about 3 to 12% (w/w) of citric acid.

6. The oral film dosage form of claim 5 wherein the dosage surface to volume ratio is between 40 to 1000.

7. The oral film dosage form of claim 5 wherein the dosage surface to volume ratio is between 50 to 500.

8. An unbuffered oral film formulation comprising
a layer comprising:
from 20 to 40% (w/w) of an antihistamine;
from 60 to 45% (w/w) a film former polymer having selected from hydroxypropyl cellulose, hydroxypropyl methyl cellulose, hydroxyethyl cellulose, carboxymethyl cellulose, methyl cellulose, polyvinylpyrollidone, pectin, gelatin, sodium alignate, polyvinyl alcohol, maltodextrins, Eudragit, and pullulan; and
from about 3 to 12% (w/w) of citric acid.

9. The oral film dosage form of claim 8 wherein the dosage surface to volume ratio is between 40 to 1000.

10. The oral film dosage form of claim 8 wherein the dosage surface to volume ratio is between 50 to 500.
